(19) United States
(12) Patent Application Publication
(10) Pub. No.: US 2007/0255336 A1
(43) Pub. Date: Nov. 1, 2007

(54) Gastric constriction device with selectable electrode combinations

(75) Inventors: Timothy P. Herbert, Maple Grove, MN (US); Warren L. Starkebaum, Plymouth, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/414,515

(22) Filed: Apr. 28, 2006

(51) Int. Cl. A61N 1/00

(52) U.S. Cl. 607/40

(57) ABSTRACT

This disclosure describes an implantable medical device that delivers electrical stimulation to a patient in combination with limiting ingestion of food by the patient to treat obesity. The device includes a gastric constriction device, such as a hydraulic or electro-mechanical gastric band, having a plurality of electrodes integrally formed thereon. One or more of the electrodes, i.e., an electrode combination, are selected to deliver electrical stimulation energy, e.g., in the form of stimulation pulses, to the patient. The electrode combination may deliver pulses in accordance with various modes, e.g., continuously, in a series of bursts, or a combination of both. When more than one electrode combination is selected, each electrode combination may deliver pulses in accordance with a different set of stimulation parameters. The electrode combinations may deliver pulses at the same time or on a time-interleaved basis.

### TECHNICAL FIELD

The invention relates to medical devices and, more particularly, to devices for the treatment of obesity.

### BACKGROUND

Various surgical techniques have been developed to treat morbid obesity. One of these techniques involves use of a gastric banding device. Gastric bands are typically constructed in the form of a hollow tube that can be inserted through a laparoscopic cannula to completely encircle an upper end of the stomach. The band is constricted to limit the passage of food into the lower stomach.

There are two basic types of gastric bands: hydraulic bands and mechanical bands. With a mechanical gastric band, the degree of gastric constriction is adjusted mechanically by a motor that tightens or loosens the band about the stomach. A hydraulic band is typically fabricated from an elastomer, such as silicone rubber. The degree of gastric constriction (the surface of the band) depends upon the amount of fluid injected into the hydraulic band. For a hydraulic band, a fluid reservoir contains an amount of fluid. A hypodermic needle may be used to percutaneously inject and withdraw fluid to and from the reservoir.

Alternatively, a pump unit may be implanted within the patient. The pump unit pumps fluid from the reservoir to the band to reduce the size of the stoma opening, and pumps fluid from the band to the reservoir to enlarge the size of the stoma opening. For a hydraulic band, a control unit implanted within the patient controls the pump and thus the size of the stoma opening. For a mechanical pump, an implanted control unit controls the motor to tighten and loosen the mechanical band.

Electrical stimulation of the gastrointestinal tract also has been used to treat obesity. Typically, electrical stimulation involves the use of electrodes implanted in the wall of a target organ, such as the stomach. The electrodes are electrically coupled to an implanted or external pulse generator via implanted or percutaneous leads. The pulse generator delivers a stimulation waveform via the leads and electrodes. For example, electrical stimulation of the stomach may be effective in reducing the desire of the patient to eat by inducing a feeling of fullness or nausea. Alternatively, electrical stimulation of the small intestine may be effective in reducing food absorption by moving the food through the small intestine more quickly, i.e., increasing gastric motility.

### SUMMARY

In general, the invention is directed to an implantable medical device that restricts ingestion of food by a patient and delivers electrical stimulation to the patient via one or more selected electrodes. The implantable medical device includes a gastric constriction device, such as a hydraulic or mechanical gastric band, and an array of electrodes integrally formed in the gastric constriction device. An implantable motor or pump may be provided to adjust the gastric constriction device to restrict food intake. An implantable pulse generator delivers stimulation energy via one or more of the electrodes integrated in the constriction device to induce a sensation of fullness or nausea.

The integration of an array of stimulation electrodes within a gastric constriction device permits a clinician to select a combination of gastric constriction and electrical stimulation to treat obesity. The implantable pulse generator may be programmed to drive a selected combination of electrodes from the integrated electrode array, or multiple electrode combinations on a time-interleaved or sequential basis. The electrodes are distributed at various positions around the gastric constriction device, permitting the clinician to test stimulation at different stimulation sites and select the most effective electrode combination or combinations. In some embodiments, additional electrodes may be provided outside of the constriction device.

In one embodiment, the invention provides an implantable medical device comprising a gastric constriction device positioned to constrict a portion of a gastrointestinal tract of a patient, a plurality of electrodes carried by the gastric constriction device, a stimulation generator that generates electrical stimulation energy, and a switch device that selects one or more of the electrodes and couples the stimulation energy to the selected electrodes to deliver the stimulation energy to the patient.

In another embodiment, the invention provides a method comprising constricting a portion of a gastrointestinal tract of a patient using a gastric constriction device, wherein the gastric constriction device carries a plurality of electrodes, and delivering electrical stimulation energy to the constricted portion of the gastrointestinal tract via a selected subset of the electrodes.

In an additional embodiment, the invention provides a device comprising means for constricting a portion of a gastrointestinal tract of a patient, wherein the constricting means carries a plurality of electrodes, and means for delivering electrical stimulation energy to the constricted portion of the gastrointestinal tract via a selected subset of the electrodes.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating an example implantable system for delivering electrical stimulation to a patient in combination with gastric banding.

FIG. 2 is a lengthwise cross-sectional side view of the gastric constriction device of FIG. 1.

FIG. 3 is a top view of the gastric constriction device of FIG. 1 in a ring configuration.

FIGS. 4A-4D are plan views of an interior side of the gastric constriction device of FIG. 2, illustrating various example electrode patterns.

FIG. 5 is a block diagram illustrating an example control unit and implantable pulse generator (IPG) of the system.

FIG. 6 is a block diagram illustrating an example external programmer in wireless communication with the gastric constriction device of FIG. 1 that allows a patient or clinician to control delivery of electrical stimulation, the degree of gastric constriction, or both.

FIG. 7 is a schematic diagram illustrating an additional example implantable system for delivering electrical stimulation to a patient in combination with gastric banding.

FIG. 8 is a schematic diagram illustrating a further example implantable system for delivering electrical stimulation to a patient in combination with gastric banding.

FIG. 9 is a flow chart illustrating a technique for delivering electrical stimulation to a patient in combination with gastric banding.

### DETAILED DESCRIPTION

Obesity is an increasing problem for many people, as individuals are consuming more calories and exercising less frequently than necessary to maintain body weight. In some cases, traditional methods for reducing body weight in obese patients may be ineffective, impractical, or potentially dangerous. In accordance with an embodiment of the invention, an implantable medical device includes a gastric constriction device, such as a hydraulic or mechanical gastric band, and an array of electrodes integrally formed in the gastric constriction device. The implantable motor or pump may be provided to adjust the gastric constriction device to restrict food intake. The implantable pulse generator delivers stimulation energy via one or more of the electrodes integrated in the constriction device to reduce appetite and/or induce a sensation of fullness or nausea.

The gastric constriction device restricts the ingestion of food to reduce caloric intake by forming a stoma opening in the stomach by encircling and partitioning the stomach into an upper and a lower stomach. Delivering electrical stimulation to the patient via selected electrodes integrally formed with the gastric constriction device may also be effective in reducing the desire of the patient to eat and prolonging a feeling of satiety in the patient in response to food intake. Stimulation may modulate or disrupt the normal myoelectric activity of the stomach or small intestine depending on where stimulation electrodes are placed and the stimulation parameters utilized. Changes in myoelectric activity may result in changes in gastric distention or gastric emptying, or in the case of the small intestine, changes in the rate at which food contents move through the small intestine. These effects, i.e., changes in myoelectric or gastrointestinal (GI) motor activity, are interpreted by the brain as feelings of early satiety, reduced appetite, or mildly unpleasant upper GI symptoms such as nausea. Nausea or other mildly unpleasant upper GI symptoms may be intentionally induced to produce aversive consequences to overeating or other dyspeptic symptoms. Changes in myoelectric or gastrointestinal motor activity, singly or in combination, may lead to reduced food intake and increase satiety by the patient, and over time, reduced body weight. Electrical stimulation may alternatively or additionally be formulated to vary gastric motility, i.e. increase gastric motility to reduce food absorption by moving the food through the gastrointestinal tract more quickly or delay gastric emptying so the patient experiences a sensation of fullness or nausea more quickly. In this manner, a gastric constriction device with integrated electrical stimulation electrodes may more completely treat obesity by limiting food intake and varying gastric motility, providing a multi-pronged therapy for treatment of obesity.

The gastric constriction device delivers electrical stimulation to the restricted portion of the gastrointestinal tract via one or more electrodes selected from a plurality of electrodes integrally formed with the gastric constriction device. The electrodes may be molded into the gastric constriction device such that each electrode has at least a partially exposed surface that contacts the patient when the gastric constriction device is implanted. The electrodes may be positioned circumferentially around the restricted portion of the gastrointestinal tract with even or irregular spacing, and are coupled to an implantable pulse generator (IPG) implanted within the patient via corresponding electrode leads. The IPG may include a switch matrix to select one or more of the electrodes to deliver electrical stimulation to the patient.

A clinician may test all or at least a portion of the possible electrode combinations of electrodes within the electrode array embedded in the constriction device in order to identify an efficacious combination of electrodes and associated polarities. An electrode combination refers to a subset of electrodes and the polarities of electrodes in the selected subset. A single electrode combination may include a number of adjacent electrodes that deliver electrical stimulation to a localized region, or electrodes arranged in a staggered configuration that deliver electrical stimulation to a more general region. Each electrode combination must include at least one anode and one cathode. In some embodiments, however, the IPG housing may function as an electrode, providing a unipolar arrangement.

More than one electrode combination may be selected to deliver electrical stimulation to the patient. In this case, multiple combinations of electrodes may be used on a time-interleaved or sequential basis to deliver stimulation to different stimulation sites. In addition, multiple stimulation programs may be delivered via one or more electrode combinations. A stimulation program generally refers to an electrode combination and a set of stimulation parameters including, for example, current or voltage amplitude, stimulation pulse width, and stimulation pulse rate. As mentioned previously, additional electrodes may be implanted independently of the gastric constriction device elsewhere in the gastrointestinal tract, e.g., in the upper stomach, lower stomach, small intestine, and/or duodenum. For example, the constriction device may be positioned about the proximal stomach, and a pair of stimulation electrodes may be positioned in the distal stomach (antrum) a few centimeters proximal to the pylorus. In this case, the constriction device serves to limit food intake, and stimulation of the antrum using suitable stimulator parameters can delay or retard gastric emptying and result in a prolonged sensation of fullness, leading to reduced food intake and eventual weight loss. As another example, applying stimulation to the proximal stomach may distend the stomach in the fasted state, thereby causing a feeling of fullness prior to meals. Consequently, applying stimulation at various locations in the gastrointestinal tract may reduce appetite, prolong satiety, or both thereby further enhancing or promoting weight loss, and enhancing the effect of a constriction device.

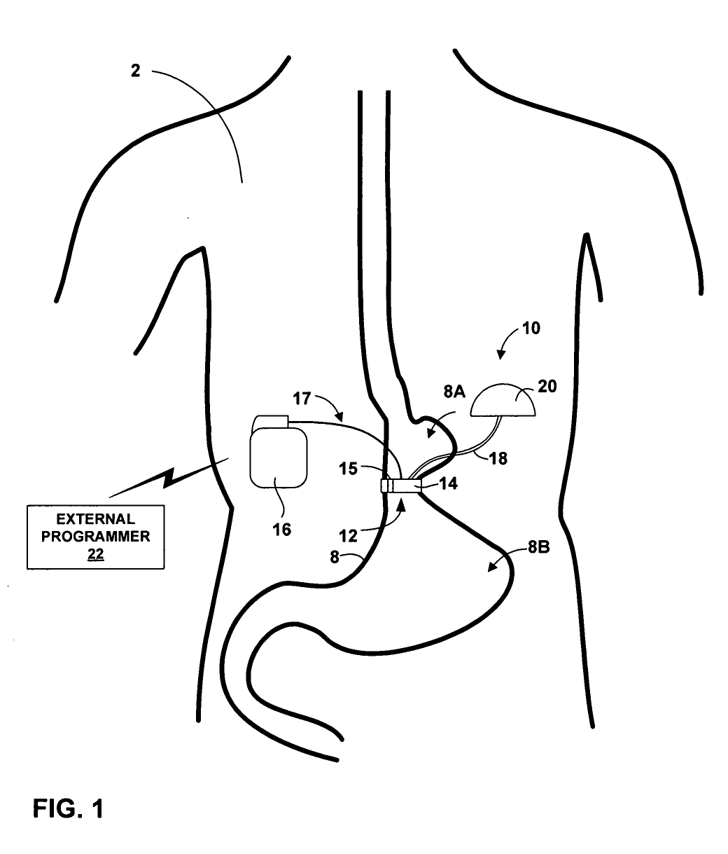

FIG. 1 is a schematic diagram illustrating an implantable medical system 10 configured for the treatment of obesity. In particular, FIG. 1 illustrates system 10 implanted within a torso of a patient 2 in which stomach 8 is visible. System 10 includes a gastric constriction device 12 with electrodes 14 (not shown) integrally formed thereon, an implantable pulse generator (IPG) 16 that generates electrical stimulation pulses, a control unit 20 for controlling the degree of gastric constriction provided by gastric constriction device 12, and an external programmer 22. System 10 treats obesity by controlling the degree of gastric constriction using gastric constriction device 12, and delivering electrical stimulation to patient 2 via selected electrodes 14 integrated with the gastric constriction device.

As shown in FIG. 1, gastric constriction device 12 forms a stoma opening in stomach 8 by encircling and partitioning stomach 8 into an upper stomach 8A and a lower stomach 8B. The degree of gastric constriction provided by gastric constriction device 12 (and thus the size of the stoma opening) is designed to limit the ingestion of food and reduce caloric intake so that patient 2 loses weight while permitting the ingestion of water and the minimum amount of caloric energy necessary to prevent malnourishment.

In addition to or, more particularly, in combination with limiting food intake, electrodes 14 (not shown) deliver electrical stimulation to patient 2 to complement or enhance the effect of constriction device. Electrical stimulation may, for example, induce a feeling of reduced appetite or fullness even in the fasted state, resulting in reduced desire by the patient to eat. In addition, electrical stimulation may be effective in reducing food absorption by increasing small intestine motility, i.e., the rate at which food moves through the small intestine or elsewhere in the gastroesophageal tract. Furthermore, electrical stimulation may be effective in decreasing gastric motility, i.e., delaying gastric emptying, so patient 2 experiences a sensation of fullness or nausea more quickly or for a prolonged period of time. Delaying gastric emptying may be achieved, for example, by increasing pyloric sphincter pressure. Delaying gastric emptying may induce a sensation of fullness or nausea more quickly than can be achieved by only ingesting food because food ingested by patient 2 does not move toward the exit of stomach 8 as quickly and, therefore, fills upper stomach 8A at an increased rate. For the same reason, patient 2 may experience a sensation of fullness for a prolonged period of time, i.e., because ingested food is delayed from exiting stomach 8. Consequently, system 10 may provide for multiple approaches for treating obesity by limiting food intake and varying gastro intestinal motility.

Although gastric constriction device 12 is shown in FIG. 1 positioned around the top end (fundus) of stomach 8 in a position commonly associated with an adjustable gastric banding (AGB) procedure, the band may also be placed vertically, as for a vertical banded gastroplasty (VBG), or in any other position designed to reduce food intake. The band may also be used with other portions of the gastrointestinal (GI) tract, such as the esophagus or intestines.

Gastric constriction device 12 may be any type of gastric constriction device, such as a hydraulic gastric band, an electro-mechanical gastric band, or another type of gastric constriction device designed to restrict or limit food intake by constriction of the stomach. Control unit 20 may be any combination of electrical circuitry and/or mechanical hardware designed to adjust the degree of constriction applied by constriction device 12.

For example, when gastric constriction device 12 comprises a hydraulic gastric band, the degree of gastric constriction, i.e., the surface of the band, depends upon the amount of fluid, such as saline or an expandable fluid, injected into the band. Accordingly, control unit 20 includes a fluid reservoir and an injection port for injecting or withdrawing fluid from the reservoir by inserting a needle into the injection port. In this case, adjustment of the band requires puncture of the patient's skin resulting in discomfort for the patient and an increased risk of infection. In order to eliminate additional medical visits and discomfort, control unit 20 may comprise a pump unit to hydraulically tighten and loosen the band.

When control unit 20 includes a pump unit, the pump unit pumps fluid from the reservoir through a conduit 18 to the band to reduce the size of the stoma opening. The pump unit may also pump fluid from the gastric band back to the reservoir to enlarge the size of the stoma opening. Thus, the degree of gastric occlusion provided by the band can be adjusted by varying the amount of fluid in the band without requiring a medical visit. In some embodiments, gastric constriction device 12 may dynamically adjust the degree of gastric constriction based on a sensed physiological parameter.

When gastric constriction device 12 is implemented as an electro-mechanical gastric band, the degree of constriction may be adjusted mechanically by means of a micro motor (not shown). The micro motor may be embedded within gastric constriction device 12 or control unit 20. For example, a micro motor may be designed to adjust the degree of constriction provided by an electro-mechanical gastric band, such as a telemetric adjustable gastric banding device. A telemetric adjustable gastric band device may enable an obstruction of the stoma to be removed without using an invasive procedure to deflate the band or endoscopy to remove the obstruction. Gastric constriction device 12 may also be any other type of mechanically adjustable gastric band. In any case, control unit 20 includes circuitry designed to control the micro motor.

A gastric band used in constriction device 12 may be constructed in the form of a hollow tube that can be inserted through a laparoscopic cannula to completely encircle the upper end of the stomach and thus restrict the passage of food into the lower stomach. The gastric band generally may be fabricated from an elastomer, such as a medical grade silicone polymer or other suitable elastomer. In the example of FIG. 1, gastric constriction device 12 comprises a hollow tube having a first end, a second end, and a connection mechanism 15 that connects the first end and the second end such that gastric constriction device 12 forms a stoma opening in stomach 8. However, the illustrated example is merely exemplary and should not be considered limiting of the invention as broadly embodied and described in this disclosure.

Gastric constriction device 12 includes a plurality of electrodes 14 (not shown) for delivering electrical stimulation to patient 2. As will be described, one or more of electrodes 14 are selected to deliver electrical stimulation to patient 2 at a given time. In any case, electrodes 14 are integrally formed with gastric constriction device 12 such that electrodes 14 are positioned circumferentially around the restricted portion of stomach 8, e.g., with regular or irregular spacing. Specifically, electrodes 14 may be molded into gastric constriction device 14 such that each of electrodes 14 has at least a partially exposed surface that contacts patient 2 when gastric constriction device is implanted within patient 2. Electrodes 14 may be integrally formed with gastric constriction device 14 using manufacturing techniques or processes similar to the techniques used to fabricate an implantable lead carrying a plurality of electrodes.

Electrodes 14 are coupled to implantable pulse generator (IPG) 16 implanted within patient 2. IPG 16 generates electrical stimulation pulses and lead 17 carries the electrical stimulation pulses to electrodes 14, i.e., electrodes 14 are electrically coupled to IPG 16 via lead 17. For purposes of illustration, only a single lead is shown in FIG. 1. However, one or more leads may carry the electrical stimulation pulses to electrodes 14. Lead 17 carries a plurality of electrical conductors. Each of the conductors is electrically coupled, at one end, to a switch matrix within IPG 16 and, at another end, to one of electrodes 14.

IPG 16 may be constructed with a biocompatible housing, such as titanium, stainless steel, or a polymeric material, and is surgically implanted within patient 2. The implantation site for IPG 16 may be a subcutaneous location in the side of the lower abdomen or the side of the lower back. IPG 16 is housed within the biocompatible housing, and includes components suitable for generation of electrical stimulation pulses. Lead 17 is flexible, electrically insulated from body tissues, and terminated with electrodes 14 integrally formed within gastric constriction device 12.

IPG 16 generates electrical stimulation pulses in accordance with a set of stimulation parameters. Thus, electrical stimulation pulses are characterized by stimulation parameters, such as voltage or current amplitude, pulse rate, pulse width, and electrode polarity. Stimulation may be provided as a continuous stream of pulses, or in bursts of stimulation pulses. Stimulation may remain on continuously 24 hours per day, or may be tuned on or off at preselected time of the day, or on the basis of one or more sensed physiological parameters. The stimulation parameters may be selected to suppress appetite in patient 2, e.g., by inducing a sensation of fullness or nausea. Alternatively or additionally, the stimulation pulses may be generated by IPG 16 to vary gastric motility. In one example, the stimulation pulses generated by IPG 16 may be selected to increase gastro intestinal motility. In particular, the stimulation pulses may cause the smooth muscle of duodenum and small intestine to contract and move contents toward the colon at an increased rate. In another example, the stimulation pulses generated by controller 16 may be configured to delay gastric emptying, e.g., by preventing the smooth muscle of stomach 8, such as, the antrum, to contract or by disrupting the coordination of smooth muscle contraction and move contents from the entrance toward the exit of stomach 8. A combination of electrical stimulation to increase gastric motility in one region of the gastrointestinal tract and decrease gastric motility in a different region of the gastrointestinal tract may also be used.

IPG 16 selects one or more of electrodes 14 as an electrode combination to deliver the electrical stimulation pulses to patient 2. Again, an electrode combination refers to the subset of electrodes selected from electrodes 14 and the polarities of the selected electrodes. An electrode combination may form one or more pairs of bipolar or multipolar electrode arrays. Alternatively, IPG 16 may carry a reference electrode to form an “active can” arrangement in which electrodes 14 are unipolar electrodes referenced to the electrode on IPG 16. Thus, a variety of polarities and electrode arrangements may be used.

For example, an electrode combination may include every other one of electrodes 14, i.e., a staggered or alternating configuration. Such an electrode combination enables electrical stimulation to be evenly delivered around the restricted portion of stomach 8. Alternatively, an electrode combination may include a number of adjacent electrodes, thereby enabling electrical stimulation to be delivered to a localized region. In this case, the electrode combination may be selected to stimulate a nerve site adjacent to the restricted portion of stomach 8, such as the vagus nerve or nerves that cause stomach 8 to contract and move food through stomach 8.

In addition, an electrode combination may deliver electrical stimulation in a variety of different modes, such as a continuous mode, in a series of bursts, or a combination of both. In some cases, rather than continuously delivering electrical stimulation over the course of a day, electrical stimulation may only be delivered over specific time intervals during the day. For example, electrical stimulation may be delivered in coordination with a specific event, such as during meal times or a sensed physiologic event. Electrical stimulation may, however, be delivered in a variety of different modes over a specific time period. In some cases, electrical stimulation may be suspended during times at which the patient is sleeping. Alternatively, stimulation may be delivered on a full-time basis.

More than one electrode combination may be used to deliver electrical stimulation to patient 2. In such embodiments, a first electrode combination may deliver electrical stimulation in accordance with a first set of stimulation parameters and a second electrode combination may deliver electrical stimulation in accordance with a second set of stimulation parameters. The first and second electrode combinations may deliver electrical stimulation at the same time or on a time-interleaved basis. For time-interleaved delivery, stimulation pulses may be delivered in an overlapping or non-overlapping manner, such that stimulation pulses delivered to different selected electrode sets are delivered in respective overlapping or non-overlapping time slots. In any case, the effect resulting from electrical stimulation, i.e., suppressing the appetite of a patient or increasing gastric motility, depends on the positions and polarities of the electrodes and the parameters associated with the stimulation pulses.

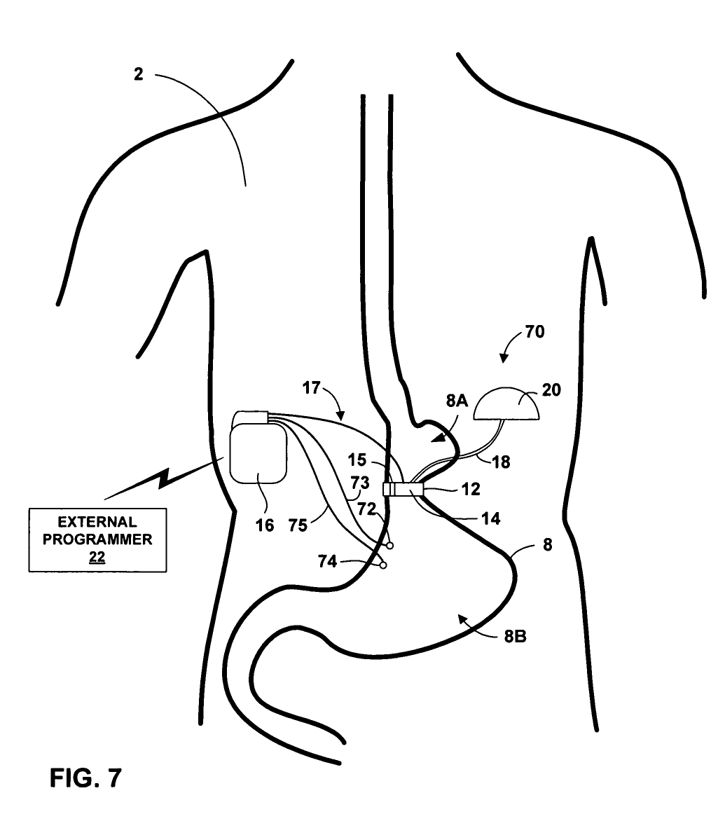

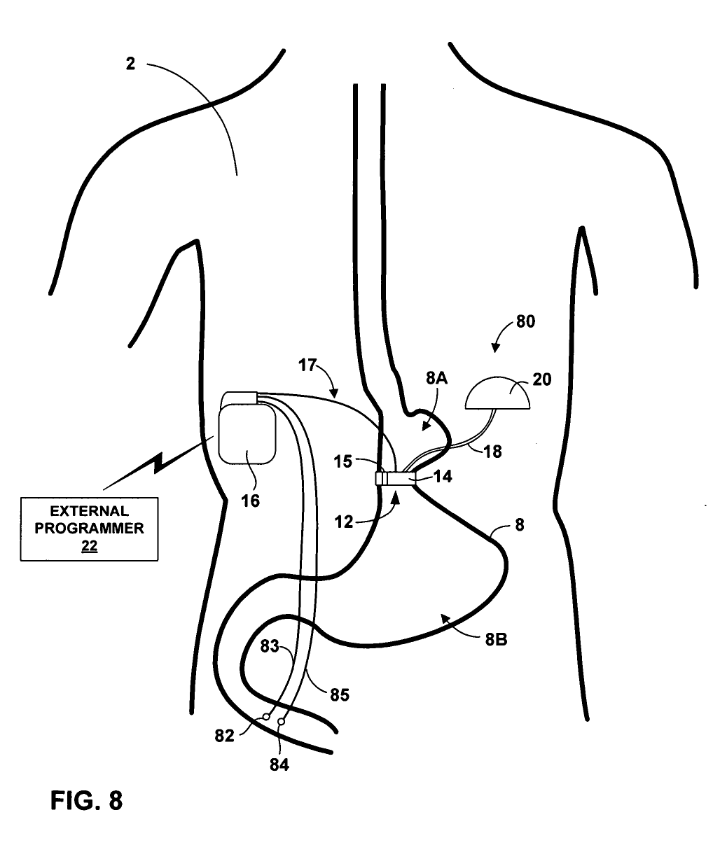

In some embodiments, electrical stimulation pulses may be delivered to other areas within the gastrointestinal tract, such as the upper stomach, lower stomach, esophagus, duodenum, small intestine, or large intestine, in addition to the restricted portion of stomach 8. In such embodiments, electrodes (not shown) may be implanted at the target organ/location and coupled to implantable stimulation via corresponding leads (not shown). For example, FIGS. 7 and 8 illustrate electrodes implanted at the stomach and duodenum, respectively, in combination with system 10. Hence, an IPG may be coupled to deliver stimulation energy to electrodes within a gastric band as well as electrodes outside of the gastric band. Delivering electrical stimulation at other areas within the gastrointestinal tract may further enhance gastric motility or suppress the appetite of the patient.

A clinician may test all or at least a portion of the possible electrode combinations of electrodes within the plurality of electrodes in order to identify an effective combination of electrodes and their polarities. Efficacy may be judged in terms of therapeutic effect in suppressing appetite, reducing food intake (liquid or solid), or by modifying (increasing or decreasing) gastric motility, gastro intestinal myoelectric activity, and in terms of the absence of undesirable side effects. Undesirable side effects may be evaluated by monitoring heart rate variability, changes in plasma hormone levels, and brain imaging. Efficacy also may be judged in terms of power efficiency provided by the selected electrode combination, particularly in light of the limited battery resources that may be available within an IPG.

The process of selecting values for the stimulation parameters that provide adequate results may be time consuming and require substantial trial and error before an effective program is identified. A clinician may need to test all possible electrode combinations or a significant portion thereof in order to identify an effective electrode combination. Consequently, in some cases, the clinician may test electrode combinations by manually specifying each combination to test based on intuition or some idiosyncratic methodology, and recording notes on the efficacy and side effects of each electrode combination after delivery in order to later compare and select from the tested electrode combinations.

The magnitude of such a task may quickly become too time consuming and costly as the number of electrodes 14 integrally formed with gastric constriction device 12 increases. Accordingly, IPG 16 may utilize a search algorithm to select electrode combinations to test. IPG 16 may receive input from the patient to indicate preferred electrode combinations. For example, patient 2 enter input to external programmer 22 in wireless communication with IPG 16. IPG 16 may store electrode combinations in internal memory in response to receiving input from the patient. The electrode combinations may be stored as programs in combination with stimulation parameters such as voltage or current amplitude, stimulation pulse width, and pulse rate.

IPG 16 may also include telemetry electronics to communicate with external programmer 22. External programmer 22 may be a small, battery-powered, portable device that accompanies patient 2 throughout a daily routine. External programmer 22 may have a simple user interface, such as a button or keypad, and a display or lights. External programmer 22 may be a hand-held device configured to permit activation of stimulation, selection of electrode combinations or stimulation programs, and adjustment of stimulation parameters. The stimulation parameters may be fixed or adjusted in response to patient input entered via external programmer 22. For example, in some embodiments, patient 2 may be permitted to adjust stimulation amplitude and turn stimulation on and off. Alternatively, programmer 22 may form part of a larger device including a more complete set of programming features including complete parameter modifications, firmware upgrades, data recovery, or battery recharging in the event IPG 16 includes a rechargeable battery.

External programmer 22 may also be configured to enable a clinician or patient to control the degree of constriction of gastric constriction device 12 and retrieve information stored in control unit 20. Typically, only a clinician may be permitted to change the degree of gastric constriction of gastric constriction device 12, although adjustment by a patient may be permitted in some circumstances. During an office visit, a clinician may download data stored in control unit 20 to external programmer 22. The clinician may view the information, thereby allowing the physician to assess the course of treatment and determine whether any adjustments are necessary. For example, the clinician may view data indicative of the degree of gastric constriction and determine if an adjustment is necessary. When an adjustment is desired, the clinician may program control unit 20 to reduce the degree of gastric constriction, i.e., cause gastric constriction device 12 to be tightened or loosened using external programmer 22.

Various surgical procedures may be used for implanting system 10 within patient 2. Well known open surgical procedures or laparoscopic surgical procedures for implanting gastric banding devices may be used to implant gastric constriction device 12 and control unit 20 within patient 2. IPG 16 may be implanted using well known surgical techniques for implanting an implantable medical device within a subcutaneous pocket of the lower abdomen of a patient. Implanting IPG 16 and control unit 20 may be implanted in a single procedure or separate procedures. However, in some embodiments, control unit 20 and IPG 16 may be contained within a single housing implanted within patient 2, thereby reducing the trauma to patient 2 because fewer incisions are required to implant system 10.

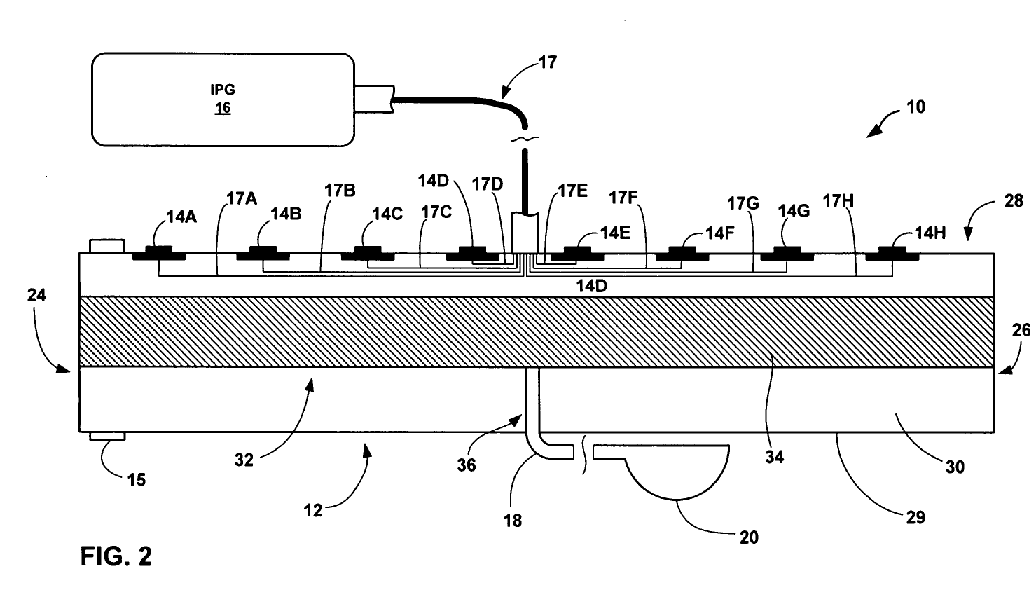

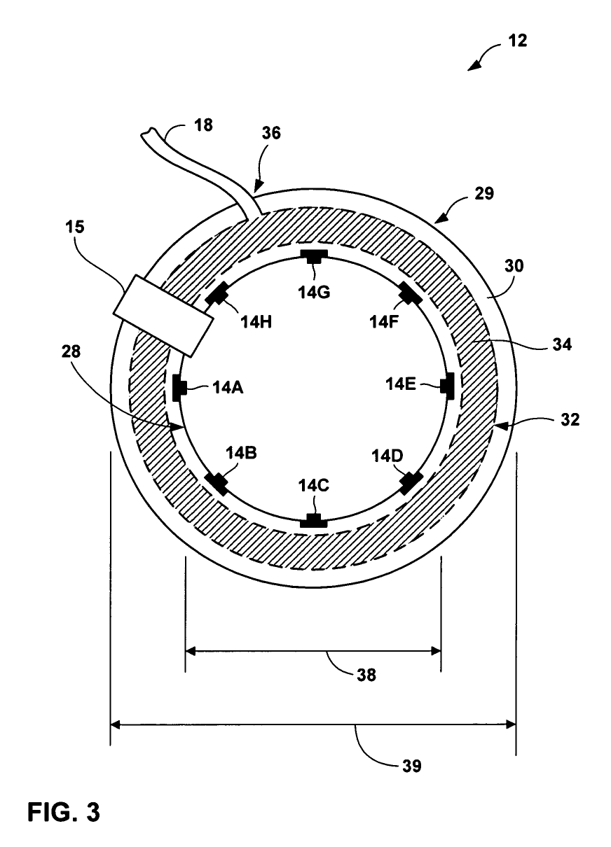

FIG. 2 is a lengthwise cross-sectional side view of gastric constriction device 12 of FIG. 1. In particular, FIG. 2 illustrates gastric constriction device 12 prior to implantation within a patient. Band 30 of gastric constriction device 12 includes an expandable lumen 32 extending longitudinally from a first end 24 to a second end 26 of band 30. When implanted within a patient, first end 24 and second end 26 are connected together via connection mechanism 15 to encircle and partition a portion of a patient's gastrointestinal tract thereby restricting ingestion of food by the patient. FIG. 3 illustrates gastric constriction device 12 connected in this manner.

In use, expandable lumen 32 is at least partially filled with a fluid 34 to restrict a portion of a patient's gastrointestinal tract. The degree of gastric constriction depends on the amount of fluid 34, e.g., saline or another fluid, within band 30 and, more particularly, lumen 32. Control unit 20 includes a fluid reservoir (not shown) and a pump unit (not shown) that pumps fluid 34 from the reservoir through conduit 18 to gastric constriction device 12. As shown in FIG. 2, control unit 20 is in fluid communication with lumen 32 via conduit 18, which enters lumen 32 through an aperture 36 in band 30. The pump unit may also pump fluid 34 from lumen 32 back to the reservoir to enlarge the size of the stoma opening.

Circuitry (not shown) within control unit 20 may control the degree of gastric constriction in response to input received from external programmer 22 (FIG. 1). Alternatively, control unit 20 may receive input from one or more sensors (not shown) implanted within the patient and control the degree of gastric constriction based on the input. For example, circuitry 20 may adjust the degree of gastric constriction in response to a sensed physiological event, such as ingestion of food. In a further embodiment, control unit 20 may adjust the degree of gastric constriction over particular time periods during the course of a day. For example, control unit 20 may increase the degree of gastric constriction by pumping fluid 34 from a fluid reservoir into lumen 32 during meal times and decrease the degree of gastric constriction by pumping fluid 34 from lumen 32 back into the reservoir at night and during the time periods between meals. Control unit 20 may also adjust the degree of gastric constriction to relieve obstruction of the stoma by food without using an invasive procedure to deflate the band or endoscopy to remove the obstruction.

Alternatively, control unit 20 may include an injection port instead of a pump unit and a fluid reservoir. In such embodiments, fluid 34 is injected or withdrawn directly from lumen 32 by percutaneously inserting a needle into control unit 20. In such embodiments, control unit 20 may be implanted just under the patient's skin. Thus, each time the degree of gastric constriction needs to be adjusted, the patient's skin must be punctured resulting in discomfort for the patient and an increased risk of infection. As a result, multiple adjustments to maintain the optimal degree of gastric constriction may be required thereby increasing the cost and number of medical visits.

In the illustrated example, electrodes 14A-H (collectively referred to as “electrodes 14”) are integrally formed with band 30 of gastric constriction device 12. In particular, each of electrodes 14 includes a portion integrally form with band 30 and an exposed surface that contacts the stomach when implanted within a patient. Electrodes 14 are electrically coupled to IPG 16 via lead 17 containing electrical conductors 17A-17H, which are coupled to respective electrodes 14A-14H. In some embodiments, each of electrodes 14 may be coupled to IPG 16 via a separate lead. However, bundling of conductors 17A-17H within a common lead 17 ordinarily will be more desirable. Conductors 17A-17H are embedded into the band 30 of gastric constriction device such that they do not contact fluid 34. For example, conductors 17A-17H may be electrically insulated and fluid sealed and/or reside within a wall of band 30, away from contact with fluid 34.

Electrodes 14 are integrally formed with band 30 such that electrodes 14 are positioned circumferentially around restricted portion of the patient's gastrointestinal tract with even spacing when implanted within the patient. Accordingly, electrodes 14 are positioned along the inner surface 28 of gastric constriction device 12 as shown in FIG. 2. By evenly spacing electrodes 14, IPG 16 can select electrode combinations to evenly distribute electrical stimulation around the restricted portion of the patient's gastrointestinal tract. In addition, a group of adjacent electrodes can be selected to deliver electrical stimulation to a localized area of the restricted portion of the gastrointestinal tract.

Alternatively or additionally, a plurality of electrodes may be similarly positioned around the outer surface 29 of band 30. By positioning electrodes around outer surface 29, electrical stimulation may be delivered to nerves proximate to the stomach, but outside the stomach wall. Stimulation of nerves proximate to stomach 8 may further induce a feeling of fullness or nausea to suppress the appetite of the patient or cause muscle of the stomach to contract and move food from the entrance of the stomach to the exit thereby enhancing gastric motility and reducing caloric absorption. As lumen 32 expands and contracts to increase or decrease inner surface 28 of gastric constriction device 12, the position of electrodes 14 may shift.

In FIG. 3, gastric constriction device 12 includes eight electrodes, i.e., electrodes 14, integrally formed with band 30 for purposes of illustration. However, gastric constriction device 12 may include a lesser or greater number of electrodes. A gastric constriction device having numerous electrodes may be particularly advantageous because the number of electrode possible combinations increases with the number of electrodes integrally formed with gastric constriction device. In other words, providing a large number of electrode combinations increases the likelihood of discovering an electrode combination that achieves a high clinical efficacy with minimal side effects and favorable power consumption characteristics.

IPG 16 includes a switch device for selecting one or more electrodes or electrode combinations to deliver electrical stimulation to the patient as previously described in FIG. 1. For example, a selected electrode combination may deliver electrical stimulation in accordance with various modes, e.g., continuously, in a series of bursts, or a combination of both. The electrode combination may also deliver electrical stimulation according to different stimulation parameters at different times during the day. When more than one electrode combination delivers electrical stimulation, each selected electrode combination may deliver electrical stimulation in accordance with a different set of stimulation parameters. The electrode combinations may deliver electrical stimulation at the same time or on a time-interleaved basis.

IPG 16 may be implanted using well known surgical techniques for implanting an implantable medical device within a subcutaneous pocket of the lower abdomen of a patient. As shown in FIG. 2, control unit 20 and IPG 16 may be implanted at different locations. Accordingly, separate incisions or possibly even separate procedures may be required to implant IPG 16 and control unit 20 within the patient. IPG 16 and control unit 20 may be implanted within the same subcutaneous pocket in order to reduce the number of incisions or procedures.

In addition, in some embodiments, IPG 16 and control unit 20 may be contained within a single housing implanted within the patient. System 10 may achieve certain benefits by enclosing IPG 16 and control unit 20 within a single housing. For example, the patient may experience less trauma, i.e., less surgery, because fewer incisions are required to implant system 10. Moreover, IPG 16 and control unit 20 may be miniaturized to fit within a single housing and, therefore, require less space.

Although a hydraulic banding device is shown in FIG. 2, gastric constriction device 12 may alternatively comprise an electro-mechanical gastric constriction device or other types of gastric constriction devices. The purpose of FIG. 2 is to illustrate the manner in which electrodes 14 are integrally formed with band 30 of gastric constriction device 12. FIG. 2 is merely exemplary and should not be considered limiting of the invention as broadly embodied and described in this disclosure.

FIG. 3 is a top view of the gastric constriction device 12 configured to restrict food intake. In particular, the ring configuration shown in FIG. 2 illustrates the configuration or shape of gastric constriction device 12 when implanted within a patient. Band 30 has an inner surface 28 and an outer surface 29 that correspond to an inner diameter 38 and an outer diameter 39. When implanted within a patient, the inner diameter 38 of band 30 determines the size of the stoma opening in the stomach. Once the desired inside surface 28 of band 30 is formed, first and second ends 24, 26 are connected together via connection mechanism 15. Connection mechanism 15 may be any type of fastening mechanism adapted to attach the two ends of band 30 together. Connection mechanism 15 may include, for example, a buckle, sutures, a clamp, adhesive, surgical staples, a coupling, or any other type of biocompatible fastener.

FIG. 3 illustrates an example configuration of electrodes 14 integrally formed with band 30 when implanted within a patient. Accordingly, electrodes 14 are positioned circumferentially along inner surface 28 with even spacing. Each of electrodes 14 has a portion integrally formed with band 30 and an exposed portion which contacts the stomach (not shown) when gastric constriction device 12 is implanted to restrict food intake of a patient. As lumen 34 expands to decrease inner diameter 38 (increase gastric constriction) and relaxes to increase inner diameter 38 (decrease gastric constriction), electrodes 14 move accordingly. In general, in embodiments where electrodes 14 are regularly spaced, electrodes 14 may remain equally spaced as the degree of gastric constriction is adjusted by control unit 20 (not shown).

Inner surface 28 may expand more easily than outer surface 29 so that inner diameter 38 can be controlled more precisely. This may be achieved by forming inner surface 28 and outer surface 29 from different materials. In this case, band 30 may be made of an inner wall and an outer wall joined together by heat-sealing, glue, solvent bonding, or mechanical means such as suturing or riveting. Thus, the inner wall and outer wall are joined to form an expandable cavity in which the outer wall expands to a substantially lesser degree than the inner wall.

As previously described, electrodes may be positioned along outer surface 29 in addition to or in place of electrodes 14. In either case, the electrodes may be positioned in a similar fashion as electrodes 14 along inner surface 28. Integrally forming electrodes along outer surface 29 may be particularly advantageous in embodiments in which outer surface is formed from a substantially non-expansible material thereby enabling the electrodes to deliver electrical stimulation to the same target area regardless of the degree of constriction of gastric banding device 12. However, electrodes integrally formed with outer surface 29 may generally be beneficial by delivering electrical stimulation to nerves proximate to the stomach wall or gastrointestinal tract of a patient.

For ease of illustration, not all of the components of gastric constriction device 12 and system 10 are shown in FIG. 3. For example, although conduit 18 is shown entering lumen 32 via aperture 36 in band 30, band control unit 20 is not shown. In addition, IPG 16 and lead 17, which electrically couples IPG 16 to electrodes 14A-14H via conductors 17A-17H, respectively, are not shown. Accordingly, FIG. 3 is merely illustrative and should not be considered limiting of the invention as broadly embodied and described within this disclosure.

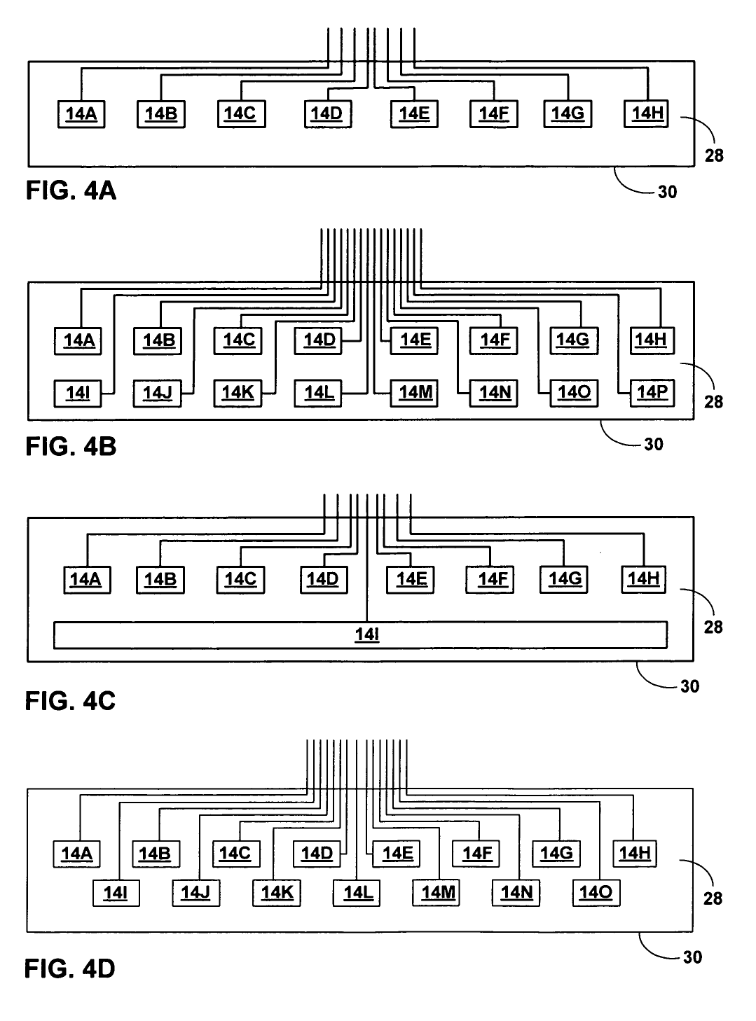

FIGS. 4A-4D are plan views of an interior side, e.g., inside surface 28, of a gastric constriction device in the form of gastric band 30 of FIG. 2, illustrating various example electrode patterns. FIG. 4A shows a linear array of electrodes 14A-14H that extend along the length of gastric band 30. In the example of FIG. 4A, electrodes 14A-14H are arrange along a common axis parallel to a longitudinal axis of band 30. Electrodes 14A-14H may be selected to form bipolar or multipolar electrode combinations. Alternatively, one electrode 14A-14H may be selected to form a unipolar combination with an electrode carried or formed by a housing of IPG 16. In either case, by selectively using one or more electrodes 14A-14H, one or more stimulation sites may be selected at different positions along the length of gastric band 30, i.e., about the periphery of the portion of the stomach constricted by the gastric band.

In the example of FIG. 4B, gastric band 30 includes two linear arrays of electrodes 14A-14H and 14I-14P that extend parallel to one another along the length of the gastric band. In FIG. 4B, electrodes 14A-14H are substantially aligned with electrodes 14I-14P, respectively, along the length of gastric band 30. One or more electrodes 14A-14H, 14A-14P in one linear array may be selected in combination with one or more other electrodes in the same linear array, or with one or more electrodes in the other linear array, or with a common electrode carried or formed by a housing of IPG 16. Although two linear arrays are shown in FIG. 4B, multiple linear arrays may be provided. In addition, such linear arrays may be arranged as multiple rows, as well as multiple columns, permitting row/column addressing to select electrodes for desired electrode combinations.

FIG. 4C shows a pattern of electrodes include a linear array of electrodes 14A-14H and a continuous electrode 14I that extends along a major portion of the length of gastric band 30. In the example of FIG. 4C, continuous electrode 14I may serve as a common electrode to form a bipolar or multipolar electrode combination with one or more of electrodes 14A-14H. In other embodiments, continuous electrode 14I may be used in combination with electrodes arranged in multiple linear arrays, e.g., on opposite sides of the continuous electrode.

In the example of FIG. 4D, gastric band 30 includes two linear arrays of electrodes 14A-14H and 14I-14P that extend parallel to one another along the length of the gastric band. In contrast to FIG. 4B, however, FIG. 4D shows the linear arrays arranged so that electrodes 14A-14H are not substantially aligned with electrodes 14I-14P, respectively, along the length of gastric band 30. Instead, electrodes 14A-14H, 14A-14P in one linear array are at staggered linear positions relative to electrodes in the other linear array. As in the example of FIG. 4B, consistent with FIG. 4D, multiple (e.g., two or more) linear arrays of electrodes may be provided in gastric band 30.

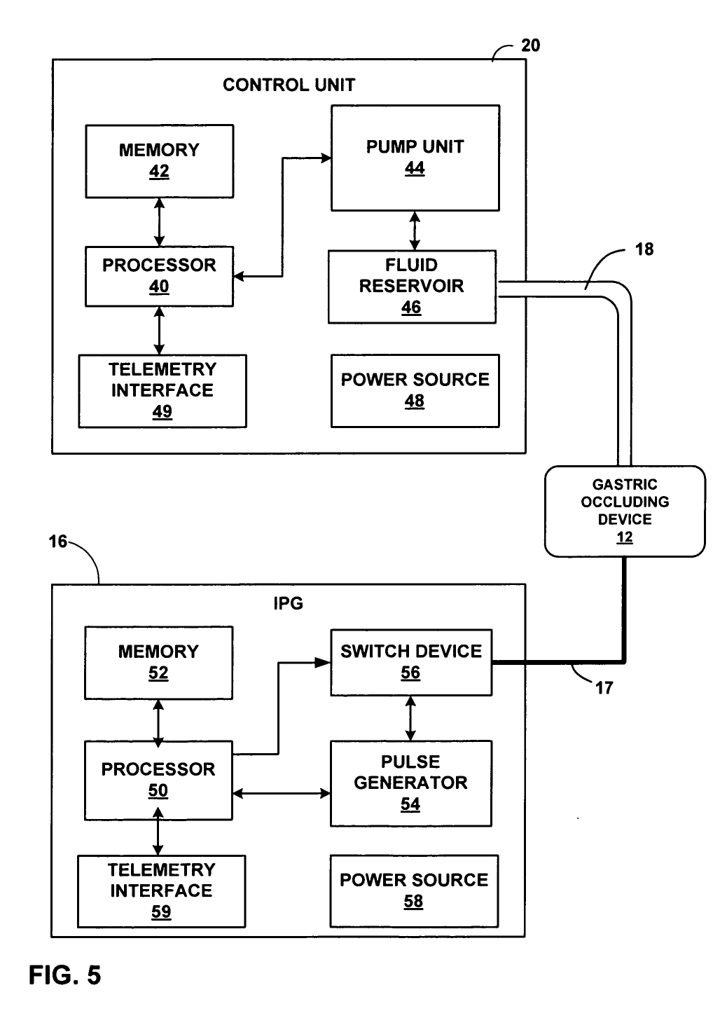

FIG. 5 is a block diagram illustrating band control unit 20 and IPG 16 of system 10. As described above, band control unit 20 hydraulically actuates a gastric constriction device 12, such as band 30, by injecting or withdrawing fluid to and from gastric constriction device 12. As shown in FIG. 5, control unit 20 may include a processor 40, which may take the form of one or more microprocessors, digital signal processors (DSPs), application specific integrated circuits (ASICs), field-programmable gate arrays (FPGAs), other discrete or integrated logic circuitry, or any combination of such components.

Control unit 20 also includes pump unit 44 which operates under the control of processor 40 to adjust the degree of gastric constriction of gastric constriction device 12. Fluid reservoir 46 contains a fluid, such as saline or another fluid, that may be injected to or withdrawn from gastric constriction device 12 to control the degree of gastric constriction. Fluid reservoir 46 may provide access for filling, e.g., by percutaneous injection of fluid via a self-sealing injection port. Fluid reservoir 46 may be contained within the housing of control unit 20 or separately.

Pump unit 44 pumps the fluid from fluid reservoir 46 and injects the fluid into an expandable lumen of gastric constriction device 12, thereby decreasing the inner diameter of device 12 and increasing the degree of gastric restriction. Pump unit 44 is in fluid communication with gastric constriction device 12 via conduit 18. Conduit 18 may comprise a flexible interconnect member, such as a catheter, that enables the transfer of the fluid between pump unit 44 and device 12. In addition, pump unit 44 can withdraw fluid from gastric constriction device 12 back to fluid reservoir 46, thereby increasing the inner diameter of device 12 and decreasing the degree of gastric restriction.

Memory 42 stores instructions that may be executed by processor 40 to control the degree of gastric constriction provided by gastric constriction device 12. Memory 42 may include a read-only memory (ROM), random access memory (RAM), electronically-erasable programmable ROM (EEPROM), flash memory, or the like. Memory 42 stores instructions that may be executed by processor 40 and thereby control the degree of gastric constriction of gastric constriction device 12. For example, processor 40 may also store data collected during treatment and/or monitoring of patient 14 within memory 42.

Memory 42 may store a schedule of times for adjusting the degree of gastric constriction and values for various degrees of gastric constriction. Processor 40 executes the instructions to cause pump unit 44 to adjust the degree of gastric constriction provided by device 12. In some embodiments, processor 40 may vary the amount of constriction over the course of a day, or adjust construction at particular time periods of the day. As an example, in some embodiments, processor 40 may cause pump unit 44 to decrease gastric constriction during preset meal times in order to allow the patient to ingest food. Processor 40 causes pump unit 44 to increase the degree of gastric constriction when it is not a preset meal time in order to limit ingestion of food by the patient. Preset meal times and values that determine the degree of constriction may be stored in memory 42 and accessed by processor 40.

Processor 40 may also store data collected during treatment and/or monitoring of a patient within memory 42. For example, in some embodiments, system 10 may include pressure sensors that generate an electrical signal indicative of the degree of gastric constriction provided by gastric constriction device 12. System 10 may also include sensors for sensing one or more physiological parameters. The sensors may be incorporated with gastric constriction device 12 or separate from device 12. In either case, processor 40 receives the signal generated by the sensor(s) and, based on the signal, controls pump unit 44 accordingly. In particular, processor 40 processes and analyzes the received signal to determine if the degree of gastric constriction needs to be adjusted. If gastric constriction needs to be adjusted, processor 40 determines the amount that the gastric constriction should be adjusted.

In some embodiments, control unit 20 may include telemetry circuitry 49, which enables processor 40 to communicate with other devices (not shown), such as an external programmer 22, via RF telemetry, proximal inductive interactive of control unit 20 with external programmer 22, or other type of wireless communication. Processor 40 controls telemetry circuitry 49 to exchange information, e.g., operational information, with external programmer 22.

The illustrated components of control unit 20 receive energy from a power source 48, such as a battery or other suitable power source. In some embodiments, power source 48 is rechargeable and power source 48 receives energy inductively captured by a recharge module (not shown). Power management circuitry (not shown) may control the recharging and discharging of power source 48. In other embodiments, power source 48 includes a nonrechargeable battery. In additional embodiments, power source 48 may receive operating power by inductive energy transfer with an external power source.

Although control unit 20 is described as hydraulically operating gastric constriction device 12, control unit 20 may alternatively mechanically operate gastric constriction device 12. In such embodiments, control unit 20 may include a micro motor that mechanically increases and decreases the inner diameter of gastric constriction device 12 to control the degree of gastric constriction instead of fluid pump 44 and fluid reservoir 46. Such a motor may wind and unwind belt or other elongated member to tighten and loosen band 30. Therefore, control unit 20 as shown in FIG. 5 should not be considered limiting to the invention as broadly embodied and described in this disclosure. Rather, control unit 20 may comprise any control electronics and devices that control the functioning, i.e., degree of gastric constriction, of a gastric constriction device.

IPG 16 controls the delivery of electrical stimulation energy to the patient via electrodes 14 integrally formed with gastric constriction device 12. As described above, electrodes 14 are positioned circumferentially around the restricted portion of stomach 8 with even spacing and deliver electrical stimulation to limit food intake and increase gastric motility. Electrodes 14 are electrically coupled to IPG 16 via lead 17, which may include a separate lead conductor for each of electrodes 14 or a bundle of conductors. In general, although eight electrodes are shown in FIGS. 2 and 3, a greater or lesser number of electrodes may be integrally formed with gastric constriction device 12 to deliver stimulation to patient 2, e.g., as shown in FIGS. 4B-4D.

In general, a relatively large number of electrodes, e.g., from eight to thirty-two, may be desirable in order to permit selection a greater number of bipolar, multipolar, and unipolar electrode combinations to deliver electrical stimulation. The availability of multiple, selectable electrode combinations increases the probability that an efficacious electrode combination will be found. In particular, a larger array of electrodes extending around the stomach permits delivery of stimulation energy to a variety of target stimulation sites on a selective basis, or delivery of stimulation energy to multiple target stimulation sites either simultaneously or on a time-interleaved basis.

As shown in FIG. 5, IPG 16 includes a processor 50, a memory 52, a pulse generator 54, switch device 56, power source 58, and telemetry circuitry 59. Memory 52 stores instructions for execution by processor 50 and stimulation parameters, such as voltage and current amplitude, pulse width, and pulse rate. Memory 52 may also record stimulation therapy data for long term storage and retrieval by patient 2 or a clinician. For example, memory 52 may store preferred electrode combinations and stimulation parameters. Alternatively, stored stimulation therapy data may be used in the adjustment of stimulation parameters. Memory 52 may include a single memory or separate memories for storing instructions, stimulation parameters sets, and stimulation information and may comprise a ROM, RAM, EEPROM, flash memory, or the like.

Processor 50 controls pulse generator 54 in delivering electrical stimulation to patient 2. Processor 50 also controls telemetry circuitry 59 in exchanging information with external programmer 22 (not shown). Based on stimulation parameters stored in memory 52 or programmed by external programmer 22, processor 50 controls pulse generator 54 and switch device 56 to deliver appropriate stimulation. As described above, processor 50 may instruct pulse generator 54 to generate electrical stimulation in accordance with various modes, e.g., continuously, in a series of bursts, or a combination of both. Additionally, each pulse may be delivered in accordance with a different set of stimulation parameters. Processor 50 may take the form of a microprocessor, DSP, ASIC, FPGA, or other equivalent integrated or discrete logic circuitry.

Pulse generator 54 comprises circuits, such as capacitors and switches, for the generation of electrical stimulation in the form of pulses. Pulse generator 54 may deliver the pulses to switch device 56, which comprises an array of switches. Processor 50 interacts with switch device 56 to select one or more electrodes for delivery of generated stimulation pulses. As previously described, processor 50 may select one or more of electrodes 14 and the polarities of each of the selected electrodes, i.e., an electrode combination, to deliver electrical stimulation to the patient. In some embodiments, processor 50 may select more than one electrode combination. In such embodiments, each electrode combination may deliver electrical stimulation in accordance with a different set of stimulation parameters. Additionally, the electrode combinations may deliver electrical stimulation at the same time or on a time-interleaved basis. In any case, based on the selected electrode combinations made by processor 50, switch device 50 delivers the pulses to the to the selected electrodes via wires of lead 17 that are electrically connect the electrodes to IPG 16.

As a further alternative, the electrode combinations may be selected so that stimulation rotates or revolves about the gastric band 30 by sequentially activating selected electrode combinations. As an illustration, if there are eight electrodes (E0 through E7) arranged linearly around the inner surface of gastric band 30, IPG 16 may sequentially activate bipolar pairs of electrodes in the following order: E0-E1, E1-E2, E2-E3, E3-E4, E4-E5, E5-E6, E6-E7. The time between activation of successive electrode pairs may be adjusted to achieve different transition rates between the electrodes.

In general, by sequentially activating electrodes that are physically positioned in a linear array around the gastric band 30, stimulation energy can be made to move around the constricted portion of the gastrointestinal tract. Stimulation can be made to move around the entire constricted portion or only a segment of the circumference of the constricted portion. In addition, stimulation may proceed around the circumference in repeated orbits in one direction, or complete one orbit or a partial orbit, and then reverse direction. Reversal of orbit direction may occur on a repetitive basis. Arrangement of electrodes on gastric band 30 permits IPG 16 to target particular stimulation sites, access multiple stimulation sites on a continuous or time-interleaved basis, or access multiple stimulation sites in sequence.

IPG 16 may also include telemetry circuitry 59, which enables processor 50 to communicate with external programmer 22 or other external devices, via RF telemetry, proximal inductive interaction with external programmer 22, or other type of wireless communication. As an example, processor 50 may control telemetry circuitry 59 to exchange information with external programmer 22. In some embodiments, processor 50 may be configured to receive instructions that control operation of IPG 16 from external programmer 22. In particular, external programmer 22 and IPG 16 may be configured to enable a clinician or patient to turn stimulation on and off or adjust stimulation amplitude or intensity using external programmer 22. Processor 50 may also transmit operational information to external programmer 22 via telemetry circuitry 59 thereby allowing a clinician to view the course of treatment and determine if adjustments are necessary.

Power source 58 delivers operating power to the components of IPG 16. Like power source 48 of control unit 20, power source 58 may include a battery or other suitable power source. In some embodiments, power source 58 is rechargeable and receives energy inductively captured by a rechargeable module (not shown). Power management circuitry (not shown) may control the recharging and discharging of power source 58. In other embodiments, power source 58 includes a nonrechargeable battery. In additional embodiments, power source 58 may receive operating power by inductive energy transfer with an external power source.

In the illustrated example, control unit 20 and IPG 16 are shown as separate modules. Accordingly, control unit 20 and IPG 16 may each be contained within a separate housing. The housing may be constructed with a biocompatible material, such as titanium, stainless steel, a polymeric material, or silicone. Alternatively, a single housing may contain control unit 20 and IPG 16 in order to reduce trauma to patient 2 during the implantation process. In this case, the electrical components of control unit 20 and IPG 16 may be mounted within a common implantable housing, and possibly on a common circuit board or boards. In some embodiments, processor 40 and processor 50 may be realized by a single, common processor. Similarly, when control unit 20 and IPG 16 are integrated as a single device, memory 42 and memory 52, telemetry interface 49 and telemetry interface 59, and power source 48 and power source 58 may be realized by common components. Because control unit 20 and IPG 16 both include a processor, memory, power source, and telemetry circuitry, the single circuit board may be miniaturized, i.e., the single circuit board may include significantly less area than two separate circuit boards.

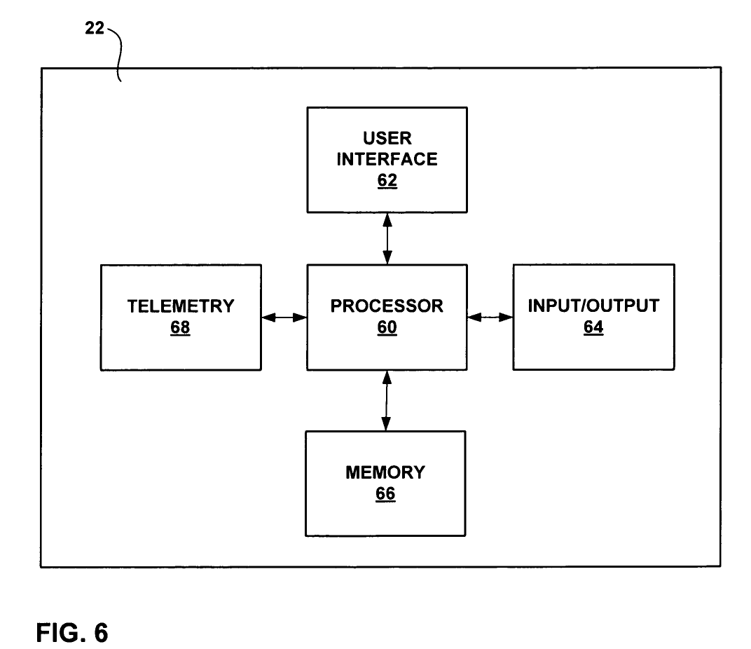

FIG. 6 is a block diagram illustrating an example of external programmer 22 in wireless communication with gastric constriction device 22. In general, external programmer 22 allows a user, such as a patient or clinician, to program or control delivery of electrical stimulation, program or control the degree of gastric constriction by gastric band 30, or both. External programmer 22 may be a small, battery-powered, portable device that accompanies patient 2 throughout a daily routine. User interface 62 may include a simple user interface, such as a button or keypad, and a display or lights. Processor 60 may also provide a graphical user interface (GUI) to facilitate interaction with the user, as will be described in detail. Processor 60 may include a microprocessor, a controller, a DSP, an ASIC, an FPGA, or other control circuitry.

External programmer 22 also includes a memory 66 that may store sets of stimulation parameters including selected electrode combinations, values for adjusting the degree of gastric constriction, and schedules for delivering electrical stimulation and adjusting the degree of gastric constriction at respective times. Generally, stored information may be available only to a clinician or other authorized user. In this manner, a clinician may program delivery of electrical stimulation by specifying parameter sets and control the degree of gastric constriction by specifying values, such as the inner diameter of gastric constriction device 12. In some cases, however, patient 2 may be permitted to adjust stimulation amplitude and/or constriction degree, and turn stimulation and/or constriction on and off.

Processor 60 transmits the selected electrode combinations, sets of stimulation parameters for deliver electrical stimulation via the selected electrode combinations, and values for adjusting the degree of gastric constriction to IPG 16 and control unit 20. Processor 60 transmits the information via wireless telemetry circuitry 68. Processor 60 also includes input/output circuitry 64 for transmitting and receiving information over a wired connection or removable electrical, magnetic, or optical media, e.g., to exchange information with another programming device.

External controller 22 may be configured to store sets of stimulation programs and program groups, and download such programs and program groups to IPG 16 when a change is requested. Alternatively, IPG 16 may store complete sets of stimulation programs and program groups, in which case external controller 22 downloads instructions for selection of one or more programs or programs groups stored in IPG 16.

In general, the term “program” may refer to a combination of parameter settings, including one or more of electrode combination, electrode polarity, pulse amplitude (current or voltage), pulse width and pulse rate, used to provide stimulation therapy. A program of stimulation therapy may be delivered alone or in combination with other programs, e.g., simultaneously via multiple stimulation channels or on a time-interleaved basis via one or more stimulation channels.

The term “group,” as used in this disclosure, may generally refer to a therapeutic stimulation therapy including one or more programs. For example, the programs in a group may be delivered, as described above, simultaneously or on a time-interleaved basis. In other words, the programs in a group of programs are delivered together in combination with one another.

FIG. 7 is a schematic diagram illustrating an example implantable system 70 configured for the treatment of obesity. Implantable system 70 includes components similar or identical to the components of system 10, but further includes electrodes 72 and 74 coupled to IPG 16 via leads 73 and 75, respectively. The components that are shared or, more specifically, common to system 10 and system 70 are identified by the same numbering in FIGS. 1 and 7. Accordingly, system 70 operates and performs in a similar fashion as system 10 but with added stimulation features because of additional electrodes 72 and 74.

In particular, by delivering electrical stimulation to lower stomach 8B via electrodes 72, 74, in addition to delivery of stimulation to the restricted portion of stomach 8 via electrodes 14 in combination with gastric banding, system 70 may more completely address or treat the factors contributing to obesity. For example, the additional electrical stimulation delivered by electrodes 72 and 74 may be selected to enhance the feeling of fullness or nausea to limit ingestion of food by patient 2 or vary gastric motility, i.e., enhance gastric motility to reduce caloric absorption from the ingested food beyond that which can be achieved by system 10, or delay gastric emptying.

In the illustrated example, leads 73 and 75 terminate into tissue of lower stomach 8B at electrodes 72 and 74, respectively. Electrodes 72 and 74 may comprise any number and type of electrodes, such as conventional ring electrode leads, paddle electrode leads, and other electrodes suitable for delivering electrical stimulation to lower stomach 8B. The stimulation pulses generated by IPG 16 cause the smooth muscle of lower stomach 8B to contract and slowly move the contents from upper stomach 8A toward the exit of lower stomach 8B. Alternatively, or additionally, the electrical stimulation pulses may stimulation nerves within lower stomach 8B to cause muscle contraction and thereby enhance gastric motility.

The electrodes carried at the distal end of each of leads 73 and 75 may be attached to the wall of lower stomach 8B in a variety of ways. For example, electrodes 72 and 74 may be surgically sutured onto the outer wall of lower stomach 8B or fixed by penetration or anchoring devices, such as hooks, barbs, or helical structures within the tissue of lower stomach 8B. Surgical adhesives may also be used to attach electrodes 72 and 74 to lower stomach 8B. In any case, electrodes 72 and 74 are implanted in acceptable electrical contact with the smooth muscle cells within the wall of lower stomach 8B. In some embodiments, electrodes 72 and 74 may be placed on the serosal surface of lower stomach 8B, within the muscle wall of stomach 8B, or within the mucosal or submucosal region of lower stomach 8B.

FIG. 8 is a schematic diagram illustrating an example implantable system 80 configured for the treatment of obesity. Similar to implantable system 70, implantable system 80, as shown, includes components similar or identical to the components of system 10 which are identified by the same numbering used in FIGS. 1 and 7. However, in contrast to system 70, system 80 includes additional electrodes 82 and 84 implanted within duodenum 86 and coupled to IPG 16 via leads 83 and 85, respectively.

In operation, implantable system 80 delivers electrical stimulation to duodenum 86 via electrodes 82, 84 in addition to restricting a portion of stomach 8 and delivering electrical stimulation to the restricted portion of stomach 8 via electrodes 14. As a result, system 80 may more completely address the contributing factors to obesity. In particular, delivering electrical stimulation to duodenum 86 may further increase gastric motility, thereby reducing caloric absorption from the food ingested by patient 2. Additionally or alternatively, delivering electrical stimulation to duodenum 86 may delay gastric emptying to induce a sensation of fullness of nausea in patient 2 more quickly. As an example, the electrical stimulation pulses generated by IPG 16 may delay gastric emptying by, for example, stimulating the pyloric sphincter (not shown).

The electrodes 82, 84 carried at the distal end of each of leads 83 and 85 may be attached to duodenum 86 in a variety of ways. For example, electrodes 82 and 84 may be surgically sutured onto duodenum 86 or fixed by penetration or anchoring devices, such as hooks, barbs, or helical structures within the tissue of duodenum 86. Surgical adhesives may also be used to attach electrodes 82 and 84 to duodenum 86. In any case, electrodes 82 and 84 are implanted in acceptable electrical contact with duodenum 86.

In some embodiments, electrical stimulation may be delivered to duodenum 86 of patient 2 via a second gastric constriction device with integrally formed electrodes. In this case, the second gastric constriction device may be implanted and function similar to gastric constriction device 12 with integrally formed electrodes 14 discussed throughout this disclosure. The electrodes of the second gastric constriction device may be coupled to IPG 16 and deliver stimulation to duodenum similar to electrodes 82, 84, i.e., in a time-interleaved or sequential manner with electrodes 14. The degree of gastric constriction of the second gastric constriction device may be adjusted to delay gastric emptying. Hence, two or more gastric constriction devices may be used at different positions in the gastrointestinal tract on a coordinated basis to retriction intake or delay emptying and apply electrical stimulation.

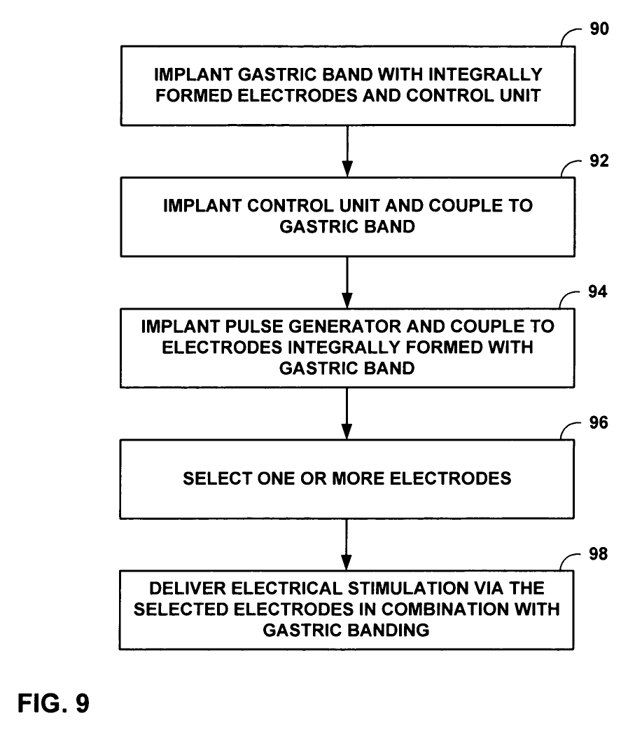

FIG. 9 is a flow chart illustrating a technique for delivering electrical stimulation to a patient in combination with gastric banding. In particular, by utilizing gastric constriction device 12 to restrict the food intake of patient 2 and deliver electrical stimulation to the restricted portion of stomach 8 via selected electrodes integrally formed with gastric constriction device 12, system 10 may limit food intake and increase gastric motility thereby providing multiple approaches for treating obesity.

Initially, gastric constriction device 12, i.e., a gastric band with a plurality of electrodes integrally formed thereon, is implanted within patient 2 (90). Typically, gastric constriction device 12 can be inserted through a laparoscopic cannula to completely encircle and partition a portion of the gastrointestinal tract into an upper and lower region thereby restricting the passage of food into the lower stomach. In some embodiments, gastric constriction device 12 may be implanted as shown in FIG. 1, although gastric constriction device 12 may be implanted at various locations of the gastrointestinal tract.

Next, various well known open or laparoscopic surgical procedures may be used for implanting control unit 20 and coupling control unit 20 to gastric constriction device 12 (92). Control unit 20 may be implanted within a subcutaneous pocket proximate to gastric constriction device 12. Control unit 20 is coupled to gastric constriction device 12 via conduit 18 so that control unit 20 and gastric constriction device 12 are in fluid communication with each other.

The surgeon may then implant IPG 16 and couple IPG 16 to gastric constriction device 12 (94) via lead 17. Because IPG 16 may be substantially similar to common IPGs used for various implantable stimulation systems and lead 17 may comprise a standard or common lead, the surgeon may use well known surgical techniques. Generally, IPG 16 may be implanted in another subcutaneous pocket in the lower abdomen of patient 2 separate from the subcutaneous pocket containing control unit 20. Accordingly, gastric constriction device 12, control unit 20, and IPG 16 may require a single or separate procedures. However, in some embodiments, control unit 20 and IPG 16 may be contained within a single housing. In this case, implanting gastric constriction device 12 and the common housing containing control unit 20 and IPG 16 may be completed in a single procedure and, thus, may reduce trauma experienced by patient 2. Furthermore, in some embodiments, electrodes may be implanted at remote locations within the gastrointestinal tract, such as the upper stomach, lower stomach, small intestines, and duodenum. As a result, the surgeon implants the electrodes at the target site, such as the lower stomach 8B or duodenum 86 as shown in FIGS. 7 and 8, respectively, and couples the electrodes to IPG 16.

When system 10, i.e., gastric constriction device 12, control unit 20, IPG 16, and any electrodes separate from electrodes 14 integrally formed with gastric constriction device 12, has been implanted within patient 2, a clinician selects one or more electrodes (96) to deliver electrical stimulation to the restricted portion of stomach 8. In general, selecting one or more electrodes includes selecting one or more of electrodes 14 or, more specifically, one or more possible electrode combinations from electrodes 14 and the stimulation parameters for delivering electrical stimulation via the selected electrode combinations.

As previously described, a clinician may test all or at least a combination of all the possible electrode combinations in order to identify an effective combination of electrodes and their polarities. In some cases, the clinician may test electrode combinations by manually specifying each combination or test based on intuition or some idiosyncratic methodology, and record notes on the efficacy and side effects of each electrode combination after delivery in order to later compare and selected from the tested electrode combinations. Alternatively, system 10 may utilize a search algorithm to select electrode combinations to test. In some embodiments, system 10 may receive input from patient 2, for example, by entering input into external programmer 22 in wireless communication with system 10, to indicate preferred electrode combinations.

When an effective, or optimum, electrode combination has been discovered, system 10 delivers electrical stimulation via the selected electrodes in combination with gastric banding (98). The selected electrodes may deliver electrical stimulation in accordance with various modes, e.g., continuously, in a series of bursts, or a combination of both. The selected electrodes may also deliver electrical stimulation according to different stimulation parameters at different times during the day or may even deliver each pulse in accordance with a different set of parameters. When more than one electrode combination is selected to deliver electrical stimulation, each selected electrode combination may deliver electrical stimulation in accordance with a different set of stimulation parameters. The electrode combinations may also deliver electrical stimulation at the same time or on a time-interleaved basis.

To induce a sensation of satiety or nausea, or modulate gastric motility, stimulation may be delivered with an amplitude of approximately 1 to 10 volts, a pulse width of approximately 0.25 to 50 milliseconds, and a pulse rate of approximately 0.05 to 40 Hz. As one example, a pulse train may be delivered according to the following stimulation parameters: amplitude approximately equal to 1 to 8 volts, pulse width approximately equal to 0.5 to 10 milliseconds, pulse rate approximately equal to 5 to 40 Hz, and ON/OFF duty cycle approximately equal to 10 to 75 percent. As another example, a series of continuous pulses may be delivered according to the following stimulation parameters: amplitude approximately equal to 1 to 8 volts, pulse width approximately equal to 1 to 20 milliseconds, pulse rate approximately equal to 0.06 to 20 Hz.

Various embodiments of the invention have been described. These and other embodiments are within the scope of the following claims.

1. An implantable medical device comprising:
a gastric constriction device positioned to constrict a portion of a gastrointestinal tract of a patient;
a plurality of electrodes carried by the gastric constriction device;
a stimulation generator that generates electrical stimulation energy; and
a switch device that selects one or more of the electrodes and couples the stimulation energy to the selected electrodes to deliver the stimulation energy to the patient.

2. The device of claim 1, wherein the gastric constriction device encircles a portion of the gastrointestinal tract and partitions the portion of the gastrointestinal tract into an upper and a lower region.

3. The device of claim 1, wherein the electrodes are integrally formed with the gastric constriction device such that the electrodes are located circumferentially around the constricted portion of the gastrointestinal tract.

4. The device of claim 1, wherein the selected one or more electrodes includes multiple electrodes.

5. The device of claim 1, wherein the electrodes are arranged in one of a linear array of electrodes that extends along a length of the gastric constriction device or a two-dimensional pattern of electrodes across a surface of the gastric constriction device.

6. The device of claim 1, further comprising a processor that controls the stimulation generator and switch device to deliver the stimulation energy to the patient in accordance with set of stimulation parameters, wherein the stimulation parameters include electrode polarity, stimulation pulse amplitude, stimulation pulse width, and stimulation pulse rate.

7. The device of claim 1, wherein the processor controls the stimulation generator and the switch device to deliver the stimulation energy to multiple selected sets of the electrodes.

8. The device of claim 7, wherein the processor controls the stimulation generator and the switch device to deliver the stimulation energy to the multiple selected sets of the electrodes on a time-interleaved basis.

9. The device of claim 1, wherein the switch device selects a first electrode combination that delivers stimulation energy to the patient and a second electrode combination that delivers stimulation energy to the patient on a time-interleaved basis with the stimulation energy delivered via the first electrode combination.

10. The device of claim 9, wherein the first electrode combination delivers stimulation energy to the patient in accordance with a first set of stimulation parameters and the second electrode combination delivers stimulation energy to the patient in accordance with a second set of stimulation parameters.

11. The device of claim 1, further comprising one or more electrodes located separately from the electrodes carried by the gastric constriction device, wherein the stimulation generator delivers stimulation energy to the gastrointestinal tract of the patient via the separately located electrodes.

12. The device of claim 11, wherein the stimulation energy delivered to the electrodes carried by the gastric constriction device is configured to induce a sensation of at least one or nausea or satiety in the patient, and the stimulation energy delivered to the separately located electrodes is configured to promote gastric motility.

13. The device of claim 1, wherein the gastric constriction device includes a hydraulic gastric band, the electrodes being integrally formed with the gastric band such at least portions of the electrodes are exposed by an interior surface of the gastric band to couple the stimulation energy to the gastrointestinal tract upon placement of the gastric band within the patient.

14. A method comprising:
constricting a portion of a gastrointestinal tract of a patient using a gastric constriction device, wherein the gastric constriction device carries a plurality of electrodes; and
delivering electrical stimulation energy to the constricted portion of the gastrointestinal tract via a selected subset of the electrodes.

15. The method of claim 14, wherein the gastric constriction device encircles a portion of the gastrointestinal tract and partitions the portion of the gastrointestinal tract into an upper and a lower region.

16. The method of claim 14, wherein the electrodes are integrally formed with the gastric constriction device such that the electrodes are located circumferentially around the constricted portion of the gastrointestinal tract.

17. The method of claim 14, wherein the selected subset of the electrodes includes multiple electrodes.

18. The method of claim 14, wherein the electrodes are arranged in a linear array of electrodes that extends along a length of the gastric constriction device.

19. The method of claim 14, wherein the electrodes are arranged in a two-dimensional pattern of electrodes across a surface of the gastric constriction device.

20. The method of claim 14, further comprising controlling the stimulation energy in accordance with set of stimulation parameters, wherein the stimulation parameters include electrode polarity, stimulation pulse amplitude, stimulation pulse width, and stimulation pulse rate.

21. The method of claim 14, further comprising delivering the stimulation energy to multiple selected sets of the electrodes.

22. The method of claim 21, further comprising delivering the stimulation energy to the multiple selected sets of the electrodes on a time-interleaved basis.

23. The method of claim 14, further comprising selecting a first electrode combination that delivers stimulation energy to the patient and a second electrode combination that delivers stimulation energy to the patient on a time-interleaved basis with the stimulation energy delivered via the first electrode combination.

24. The method of claim 23, further comprising delivering the stimulation energy to the first electrode combination in accordance with a first set of stimulation parameters and delivering the stimulation energy to the second electrode combination in accordance with a second set of stimulation parameters.

25. The method of claim 14, further comprising delivering stimulation energy to the patient via one or more electrodes located separately from the electrodes carried by the gastric constriction device.

26. The method of claim 25, wherein the stimulation energy delivered to the electrodes carried by the gastric constriction device is configured to induce a sensation of at least one or nausea or satiety in the patient, and the stimulation energy delivered to the separately located electrodes is configured to promote gastric motility.

27. The method of claim 14, wherein the gastric constriction device includes a hydraulic gastric band, the electrodes being integrally formed with the gastric band such at least portions of the electrodes are exposed by an interior surface of the gastric band to couple the stimulation energy to the gastrointestinal tract upon placement of the gastric band within the patient.

28. A device comprising:
means for constricting a portion of a gastrointestinal tract of a patient, wherein the constricting means carries a plurality of electrodes; and
means for delivering electrical stimulation energy to the constricted portion of the gastrointestinal tract via a selected subset of the electrodes.

29. The device of claim 28, wherein the constricting means includes a gastric band that encircles a portion of the gastrointestinal tract and partitions the portion of the gastrointestinal tract into an upper and a lower region.

30. The device of claim 29, wherein the electrodes are integrally formed with the gastric band such that the electrodes are located circumferentially around the constricted portion of the gastrointestinal tract.

31. The device of claim 29, wherein the selected subset of the electrodes includes multiple electrodes.

32. The device of claim 29, wherein the electrodes are arranged in a linear array of electrodes that extends along a length of the gastric band.

33. The device of claim 28, further comprising means for controlling the stimulation energy in accordance with set of stimulation parameters, wherein the stimulation parameters include electrode polarity, stimulation pulse amplitude, stimulation pulse width, and stimulation pulse rate.

34. The device of claim 28, further comprising means for delivering the stimulation energy to multiple selected sets of the electrodes on a time-interleaved basis.

35. The device of claim 28, further comprising means for selecting a first electrode combination that delivers stimulation energy to the patient and a second electrode combination that delivers stimulation energy to the patient on a time-interleaved basis with the stimulation energy delivered via the first electrode combination, and means for delivering the stimulation energy to the first electrode combination in accordance with a first set of stimulation parameters and delivering the stimulation energy to the second electrode combination in accordance with a second set of stimulation parameters.

36. The device of claim 28, further comprising means for delivering stimulation energy to the patient via one or more electrodes located separately from the electrodes carried by the gastric constriction device, wherein the stimulation energy delivered to the electrodes carried by the gastric band is configured to induce a sensation of at least one or nausea or satiety in the patient, and the stimulation energy delivered to the separately located electrodes is configured to promote gastric motility.
